(19) United States
(12) Patent Application Publication
(10) Pub. No.: US 2023/0309884 A1
(43) Pub. Date: Oct. 5, 2023

(54) PHYSIOLOGICAL MEASUREMENT DATA PROCESSING DEVICE AND PHYSIOLOGICAL MEASUREMENT DATA PROCESSING METHOD USING SAME

(71) Applicant: Hitachi, Ltd., Tokyo (JP)

(72) Inventor: Shunsuke MINUSA, Tokyo (JP)

(73) Assignee: Hitachi, Ltd., Tokyo (JP)

(21) Appl. No.: 18/166,012

(22) Filed: Feb. 8, 2023

(30) Foreign Application Priority Data
Mar. 29, 2022 (JP) 2022-053214

(51) Int. Cl. A61B 5/16; G16H 10/20

(52) U.S. Cl. CPC A61B 5/165; G16H 10/20

(57) ABSTRACT

A physiological measurement data processing device includes a reception unit receiving, from a user terminal of a user, physiological measurement data obtained by measuring a physiological state of the user with a sensor, a deviation assessment unit that assesses a deviation of the physiological measurement data received by the reception unit with respect to past physiological measurement data of the user, a subjective score assessment unit estimating a subjective score indicating a subjective state of mind and body of the user based on the physiological measurement data received by the reception unit, an uncertainty assessment unit that assesses an uncertainty of the subjective score estimated by the subjective score assessment unit, and a discrepancy assessment unit that assesses a discrepancy indicating a discrepancy regarding a usual state of the user based on the deviation assessed by the deviation assessment unit and the uncertainty assessed by the uncertainty assessment unit.

### CROSS-REFERENCE TO RELATED APPLICATION

This application claims priority to Japanese Patent Application No. 2022-053214, filed on Mar. 29, 2022, the entire contents of which are incorporated by reference herein.

### BACKGROUND OF THE INVENTION

### 1. Field of the Invention

The present invention relates to a physiological measurement data processing device used for determining a timing at which a user is in a subjective state that is different from usual, and the like, based on physiological state measured from the user, and a physiological measurement data processing method using the same.

### 2. Description of Related Art

JP-A-2016-122434 (PTL 1) discloses a home medical support system. A wearable terminal attached to the body of a subject in need of assistance collects physiological information from the body of the subject in need of assistance, and the collected physiological information is wirelessly transmitted to a receiver which is a home device and analyzed by an information analysis unit of the receiver. When the information analysis unit determines the presence of anomaly in the physiological information, the physiological information is transmitted to the medical institution without requiring the subject in need of assistance to press the doctor call button. Examples of the anomaly in the physiological information include pulse rate, blood pressure, and oxygen saturation exceeding its predetermined thresholds.

### SUMMARY OF THE INVENTION

Meanwhile, techniques are under research, which estimate physical and mental health conditions such as emotions, fatigue, and moods of users in daily life based on the data obtained by measuring physiological states. By using this technology, it is possible to determine based on physiological measurement data whether or not the user is in a mental state different from usual, and implement intervention measures such as providing mental care based on the determination results.

PTL 1 implements intervention based on the physiological information itself that is measurable by a wearable terminal. On the other hand, when considering a service that estimates the subjective state of mind and body based on the physiological information among the physical and mental health conditions, and performs intervention measures for individuals, since the relationship between the subjective state of the mind and body and the physiological state is complicated, it is difficult to implement intervention measures at an appropriate timing by simply determining the conditions based on the physiological information, and it is desirable to collect the data in the past that includes the subjective state of the mind and body of the user and the actual physiological information corresponding thereto, and build a model between the physiological information and the subjective state of mind and body by machine learning. For this purpose, it is necessary to collect physiological information for the training data when the user is in various subjective states of mind and body.

However, it is difficult to acquire high-quality training data for such subjects related to the subjectivity of the user. For example, if the subjective state of mind and body is surveyed by means of questionnaires (Experience Sampling Method (ESM) or Ecological Momentary Assessment (EMA)) at regular or random timing, it cannot be guaranteed that a change occurs in the subjective state of mind and body of the user at the timing of the questionnaire, and there also is a possibility of missing data during intense emotional experiences that occur with a low frequency of occurrence. If the user can record the subjective state of mind and body at the timing when the user is aware of the change in the subjective state of mind and body, it will be possible to capture major changes in the subjective state of mind and body, but it is not possible to capture unconscious changes in the subjective state of the mind and body.

The present invention has been made in view of the problems described above, and it is an object of the present invention to enable reports about intervention measures and data collection at appropriate timing such as when the user is in a subjective state different from usual.

A physiological measurement data processing device may include a reception unit that receives, from a user terminal of a user, physiological measurement data obtained by measuring a physiological state of the user with a sensor; a deviation assessment unit that assesses a deviation of the physiological measurement data received by the reception unit with respect to past physiological measurement data of the user; a subjective score assessment unit that estimates a subjective score indicating a subjective state of mind and body of the user based on the physiological measurement data received by the reception unit; an uncertainty assessment unit that assesses an uncertainty of the subjective score estimated by the subjective score assessment unit; and a discrepancy assessment unit that assesses a discrepancy indicating a discrepancy with respect to a usual state of the user based on the deviation assessed by the deviation assessment unit and the uncertainty assessed by the uncertainty assessment unit.

It is possible to take actions such as reporting about intervention measures, collection of ground-truth data, and the like while considering the subjective state of a user.

The details of at least one embodiment of the subject matter disclosed herein are set forth with reference to the accompanying drawings and the description below. Other features, aspects, and advantages of the disclosed subject matter will be apparent from the following description, drawings, and claims.

### BRIEF DESCRIPTION OF THE DRAWINGS

FIG. 1 is a block diagram of an example of a main configuration of a physiological measurement data processing system;

FIG. 2 is a flowchart provided to explain an example of a process of receiving from a user terminal;

FIG. 3 is a flowchart provided to explain an example of a process of training a discrepancy assessment model in subjective score estimation;

FIG. 4 is a flowchart provided to explain an example of a discrepancy assessment process in subjective score estimation;

FIG. 5A is a flowchart provided to explain an example of a report determination process based on a discrepancy;

FIG. 5B is a flowchart provided to explain an example of a process of outputting a report of an event in which reporting the event is determined based on the discrepancy;

FIG. 6 is a flowchart provided to explain an example of a process of receiving a response result in response to a report output;

FIG. 7A is an example of a report output screen displayed on a user terminal;

FIG. 7B is an example of a report output screen displayed on the user terminal;

FIG. 8 is an example of a report output screen displayed on an administrator terminal;

FIG. 9A is an example of a screen displaying subjective score estimation result displayed on the user terminal;

FIG. 9B is an example of a screen displaying subjective score estimation result displayed on the user terminal;

FIG. 9C is an example of a screen displaying subjective score estimation result displayed on the user terminal;

FIG. 10A is a diagram showing an example of a data structure of user data;

FIG. 10B is a diagram showing an example of the data structure of subjective score estimation data;

FIG. 10C is a diagram showing an example of the data structure of discrepancy data;

FIG. 10D is a diagram showing an example of the data structure of determination result data;

FIG. 10E is a diagram showing an example of the data structure of response result data;

FIG. 10F is a diagram showing an example of the data structure of user characteristic data;

FIG. 10G is a diagram showing an example of the data structure of subjective score ground-truth data;

FIG. 11A is an example of a screen displayed on a user terminal to report the need for intervention based on subjective scores;

FIG. 11B is an example of a screen displayed on the user terminal to report the need for intervention based on subjective scores;

FIG. 11C is an example of a screen displayed on the user terminal to report the need for intervention based on subjective scores;

FIG. 12A is a flowchart provided to explain an example of a training process with similar user classification;

FIG. 12B is a flowchart provided to explain an example of a similar user classification process;

FIG. 13A is an example of an output screen displayed on the user terminal; and

FIG. 13B is an example of an output screen displayed on the user terminal.

### DESCRIPTION OF EMBODIMENTS

Hereinafter, embodiments of the present invention will be described with reference to the drawings.

### First Embodiment

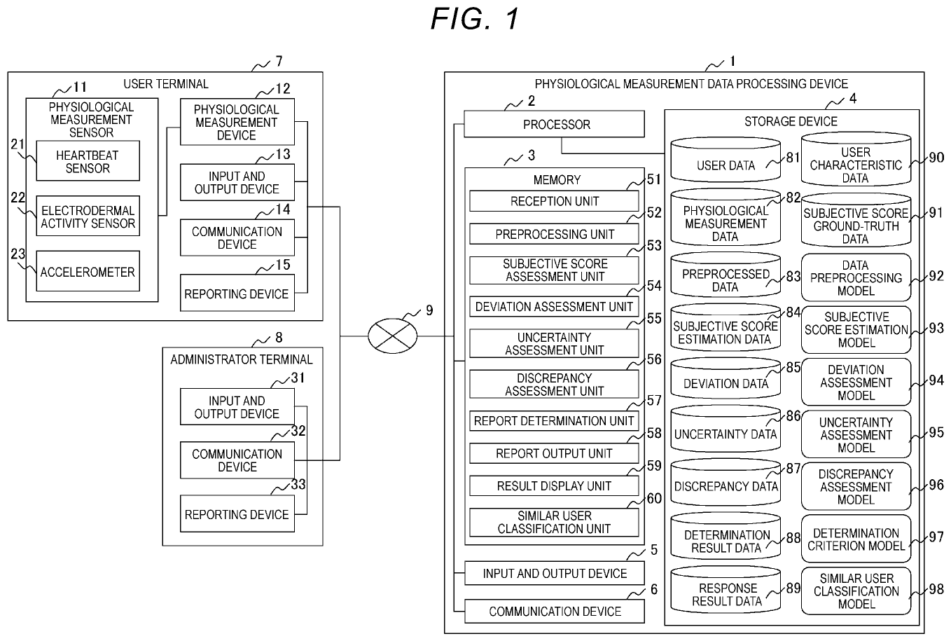

FIG. 1 is a block diagram of an example of a main configuration of a physiological measurement data processing system. The physiological measurement data processing system according to an embodiment includes one or more user terminals 7, one or more administrator terminals 8, and a physiological measurement data processing device 1 that processes data received from the user terminals 7 via a network 9.

The user terminal 7 includes a physiological measurement sensor 11 that measures a physiological state of the user, a physiological measurement device 12 that controls the physiological measurement sensor 11, an input and output device 13, a communication device 14, and a reporting device 15.

The physiological measurement sensor 11 includes a heartbeat sensor 21 that measures an interval between heartbeats of a user (R-R Interval, RRI), an electrodermal activity sensor 22 that measures an amount of perspiration of the user, and an accelerometer 23 that measures a movement of the user. For the heartbeat sensor 21, a sensor that measures heartbeats based on electrocardiogram, pulse wave, pressure change, heart sound, or the like may be used. The physiological measurement sensor 11 is not limited to the examples described above, and other sensors that measure body temperature, blinking, eye movement, myoelectricity, brain waves, or the like may be employed. As the physiological measurement sensor 11, in addition to a wearable device that can be worn on the user, a built-in system in a smart phone or the like that may be carried around by the user may be used.

The physiological measurement device 12 controls the physiological measurement sensor 11 and performs arithmetic operation and compression as necessary with respect to the physiological states measured by the physiological measurement sensor 11 to generate physiological measurement data 82.

The input and output device 13 displays a screen on the user terminal 7 and receives subjective score ground-truth data 91, i.e., the ground-truth data of the subjective state of mind and body that is to be estimated based on the physiological information.

The reporting device 15 reports to the user according to a report output from the physiological measurement data processing device 1. For example, in addition to the screen displayed on the user terminal 7 by the input and output device 13, report may be output to the user using vibration or sound.

While it is illustrated in this example that the user terminal 7 includes the physiological measurement sensor 11 and the physiological measurement device 12, the physiological measurement sensor 11 and the physiological measurement device 12 do not necessarily have to be formed as one device (hardware). For example, of the components in the user terminal 7, the input and output device 13 and the communication device 14 may be configured in a smart phone, the physiological measurement device 12, the physiological measurement sensor 11, and the reporting device 15 may be configured in a smart watch, or the smart phone and the smart watch may be treated as an integrated system in which case the user terminal 7 may be configured in the smart phone and the smart watch.

The administrator terminal 8 includes an input and output device 31, a communication device 32, and a reporting device 33. While the term “administrator” herein broadly refers to a person who is in charge of managing and supervising the user, although the relationship such as a superior to a subordinate is not particularly limited. The reporting device 33 reports to the administrator according to the report output from the physiological measurement data processing device 1. For example, in addition to the screen displayed on the administrator terminal 8 by the input and output device 31, report may be output to the administrator using vibration or sound.

The physiological measurement data processing device 1 is a computer including a processor 2, a memory 3, a storage device 4, an input and output device 5, and a communication device 6. The memory 3 loads, as programs, the functional units of each of a reception unit 51, a preprocessing unit 52, a subjective score assessment unit 53, a deviation assessment unit 54, an uncertainty assessment unit 55, a discrepancy assessment unit 56, a report determination unit 57, a report output unit 58, a result display unit 59, and a similar user classification unit 60. Each program is executed by the processor 2. Each functional unit will be described below in detail.

The processor 2 operates as a functional unit that provides a predetermined function by executing a process according to the program of each functional unit. For example, the processor 2 serves as the discrepancy assessment unit 56 by executing a discrepancy assessment program. The same applies to the other programs. Furthermore, the processor 2 also operates as a functional unit that provides respective functions of multiple processes executed by each program. The computer and a computer system are a device and a system that include these functional units.

The storage device 4 stores data used by each functional unit described above. The storage device 4 stores user data 81, the physiological measurement data 82, preprocessed data 83, subjective score estimation data 84, deviation data 85, uncertainty data 86, discrepancy data 87, determination result data 88, response result data 89, user characteristic data 90, subjective score ground-truth data 91, a data preprocessing model 92, a subjective score estimation model 93, a deviation assessment model 94, an uncertainty assessment model 95, a discrepancy assessment model 96, a determination criterion model 97, and a similar user classification model 98. Details of the data and models mentioned above will be described below.

The input and output device 5 includes an input device such as a mouse, a keyboard, a touch panel, a microphone, and the like, and an output device such as a display, a speaker, and the like. The communication device 6 communicates with the user terminal 7 and the administrator terminal 8 via the network 9.

In the embodiments described below, it will be described as an example that the user always wears or carries the user terminal 7 in daily life and the physiological measurement sensor 11 is in operation, although the present invention is not limited to this example. For example, the physiological measurement sensor 11 may be operated using the user terminal 7 only several times a day, such as when waking up or when going to bed.

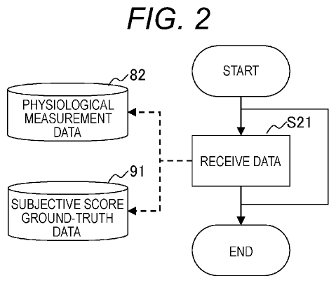

FIG. 2 is a flowchart provided to explain an example of a process of receiving from the user terminal 7, which is performed by the physiological measurement data processing device 1.

When the connection to the user terminal 7 is established via the network 9, the reception unit 51 of the physiological measurement data processing device 1 starts data reception S21. When the data reception S21 is started, the physiological measurement data processing device 1 receives physiological information, which is measured by the physiological measurement sensor 11 of the user terminal 7 and converted into data by the physiological measurement device 12, and stores the received information as the physiological measurement data 82. The data reception continues until disconnection from the user terminal 7. An example in which the heartbeat interval data, electrodermal activity data, and acceleration data are measured and stored as the physiological measurement data 82 will be described below.

Furthermore, in the input and output device 13 of the user terminal 7, when the ground-truth data is input for the subjective state of mind and body that is the data to be estimated based on the physiological information, the physiological measurement data processing device 1 receives the ground-truth data input together and stores it as the subjective score ground-truth data 91.

In this example, the subjective state of mind and body to be estimated by the system is assumed to be natural emotions in daily life. In this case, for the subjective score ground-truth data 91, it is conceivable to measure the natural emotion in the emotion dimensions including arousal and valence. In this case, the affective scale that represents and measures the emotion dimension with pictograms at both ends of a visual scale, i.e., visual analogue scale (VAS), or self-assessment manikin (SAM) that measures with multi-level pictograms may be used for measurement.

Furthermore, in addition to measuring the emotion dimension, emotional experience may be measured by using the Positive and Negative Affect Schedule (PANAS) which measures discrete emotions such as happiness and the like according to correlations to adjectives that indicate emotions.

While it is described as an example that the physiological measurement data 82 is received continuously, it is not necessarily continuous. For example, the user terminal 7 may receive the physiological measurement data 82 collected every predetermined time such as 2 minutes, 30 minutes, or the like. In addition, the connection may be established and the data reception process S21 may be performed only when the user terminal 7 side performs transmission to the physiological measurement data processing device 1.

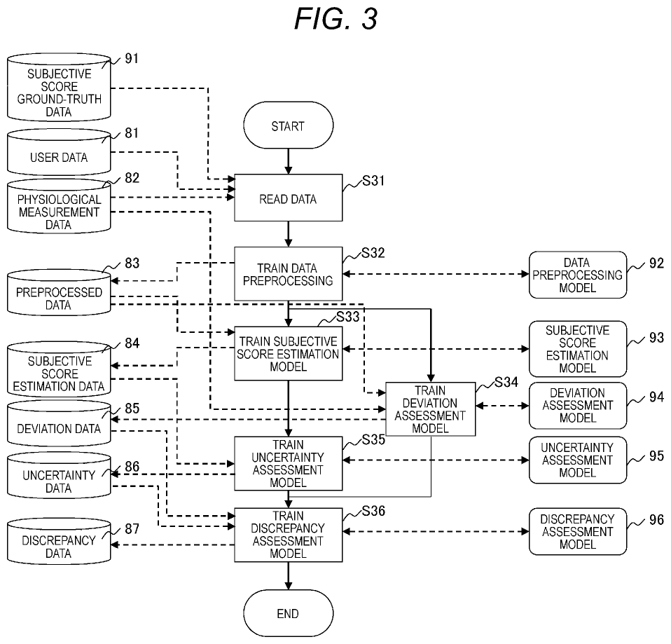

FIG. 3 is a flowchart provided to explain an example of a process of training the discrepancy assessment model 96 in subjective score estimation performed by the physiological measurement data processing device 1. First, the reception unit 51 of the physiological measurement data processing device 1 performs a data read process S31 of reading the subjective score ground-truth data 91 used for training, the physiological measurement data 82, and the user data 81 storing the information of the user from whom the physiological measurement data 82 is acquired. In addition, unless otherwise specified, it is assumed that a set of physiological measurement data 82 includes heartbeat interval data, electrodermal activity data, and acceleration data for 30 minutes, and a set of subjective score ground-truth data 91 is the strength of valence and arousal for 30 minutes, and a plurality of sets of these are used to perform the training process of the discrepancy assessment model 96.

In the training process of data preprocessing S32, the preprocessing unit 52 first uses the read user data 81 and physiological measurement data 82 to perform the training process of data preprocessing. The training process of data preprocessing involves training the data preprocessing model 92 that performs a correction process for individual variabilities included in the physiological measurement data 82, a process of extracting features from the physiological measurement data 82, and a dimensionality reduction process for the feature values extracted from the physiological measurement data 82, and the like. These processes may be explicitly sequentially executed as a pipeline by the data preprocessing model 92 that has a plurality of divided processes (subtasks), or may be processed end-to-end (at once without dividing into subtasks) using the data preprocessing model 92 that is trained with the data.

For example, a series of data preprocessing may be configured such that, a data normalization process may be performed based on a series of physiological measurement data 82 measured by the user himself or herself in the past as a correction process for individual variabilities, and the feature extraction process may be performed by a dimensionality reduction process based on principal component analysis with the physiological measurement data 82 normalized for each user as input. In this case, the normalization parameter for each user, the number of principal components of the principal component analysis, and the obtained eigenvectors may be trained by the training process of data preprocessing, and the data preprocessing model 92 may be built. In this data preprocessing model 92, the normalization parameter for each user used for the correction process for the individual variabilities may be treated as a correction model for the physical variability of the individual variabilities, and the rest of the data preprocessing model 92 excluding the physical variability correction model (normalization parameter) may be applied as a data preprocessing model that may be used commonly among multiple users, with individual variabilities due to physical characteristics between individuals removed, thereby improving robustness.

In addition, for end-to-end processing, it is also possible to set a measurement noise removal process of the physiological measurement data 82 as the data preprocessing, and use latent vectors of Variational Auto Encoder (VAE), which is a deep layer generation model, which is input with physiological measurement data 82 subjected to noise removal in the feature extraction/dimensionality reduction process. In this case, the VAE model is trained with the physiological measurement data 82 by unsupervised learning.

For the purpose of correcting individual variabilities and removing noise, a normalization process and a noise removal process of the used signal scale may be performed. For example, the normalization process may involve, for each user, each measurement day, each measurement day with a user, or between all users, the min-max normalization that normalizes by the maximum and minimum values, the z-scoring that normalizes with the mean and standard deviation of the signals, the quantile normalization that normalizes using the quantiles of the signal strength distribution, or the like. In addition, if the signal strength that varies due to aging, and the like is known, the age information of the user data 81 may be taken into account, and a deviation value conversion process for normalizing the signal strength for each age group and the like may be performed. Furthermore, the noise removal process may involve clipping or winsorize process that removes abnormal values from the signal to keep it within a certain range, or moving average process that suppresses and smoothes sudden variations at one time, or zero-order differentiation process that uses the Savitzky-Golay filter, and the like.

For the process of extracting features from the physiological measurement data 82, the feature extraction process may be performed according to the physiological signal of the physiological measurement data 82 used. For example, for the heartbeat interval data acquired by the heartbeat sensor 21, the average heart rate, the Low Frequency component (LF) and High Frequency component (HF), which are known to mainly reflect sympathetic nerve activity and parasympathetic nerve activity obtained by frequency domain analysis, respectively, the feature values by the SDNN, RMSSD, and NN50 used in time domain analysis or the Lorentz plots used in nonlinear domain analysis, the feature values obtained by Detrended Fluctuation Analysis, the feature values obtained by the complex demodulation method, and so on may be used. For the electrodermal activity data acquired by the electrodermal activity sensor 22, the skin conductance level (SCL) or the skin conductance response (SCR) may be used. For the triaxial acceleration data obtained from the accelerometer 23, the acceleration norm, the number of zero crosses, which is the number of times a signal processed by a band-pass filter with respect to the acceleration norm exceeds a threshold of ±0.01 G when the gravitational acceleration is 1 G, or the like may be used.

Further, in addition to the physiological measurement data 82, the subjective score ground-truth data 91 may be subjected to predetermined data preprocessing. For example, when estimating the strength of valence and arousal, the preprocessing may be performed according to the type of the subjective score estimation model 93 used in the training process of the subjective score estimation model which will be described below. For example, if valence and arousal are measured on a 5-step Likert scale from 1 to 5, in the case of training a regression model, it may be scaled from −1 to 1. In addition, in the case of a classification model, 1 and 2 may be binarized as a negative example 0, and 4 and 5 as a positive example 1, except for the intermediate value 3.

A known algorithm may be used for the dimensionality reduction process. For example, the principal component analysis described above, Auto Encoder (AE) as an autoencoder, Uniform Manifold Approximation and Projection (UMAP), and the like may be used.

Further, because the training process of data preprocessing is typically based on unsupervised learning, the training process may be performed with the physiological measurement data 82 that does not correspond to the subjective score ground-truth data 91. Since the subjective score ground-truth data 91 is expensive to acquire, the configuration as described above may allow the data preprocessing model 92 to be trained with a larger number of physiological measurement data 82, thereby providing an effect of training the data preprocessing model 92 that is capable of generating preprocessed data that can express more diverse states as the feature values.

When the training process of data preprocessing is completed as described above, the preprocessing unit 52 performs data preprocessing on the physiological measurement data 82 using the trained data preprocessing model 92 to generate the preprocessed data 83.

In the training process of the subjective score estimation model S33, the subjective score assessment unit 53 uses the subjective score ground-truth data 91 and the preprocessed data 83 to train the subjective score estimation model 93. For example, in order to estimate natural emotions in daily life, an emotion dimension estimation model for estimating strengths of valence and arousal for a set of subjective score ground-truth data 91 for 30 minutes is trained by supervised learning. The subjective score estimation model 93 may be configured using a known algorithm. For example, for the machine learning algorithms, logistic regression models, decision trees, Random Forests, Support Vector Machines, neural networks, Bayesian neural networks, deep learning models, and the like may be used. As for the algorithm, either a classification algorithm or a regression algorithm may be used according to the subjective score to be estimated. For example, when estimating the strength of valence and arousal from −1 to 1, a regression algorithm may be used, and when estimating the high-low of the strength of valence and arousal, a classification algorithm may be used instead of the regression algorithm.

While the training process of data preprocessing and the training process of the subjective score estimation model are described as different processes from each other in the flowchart, a configuration in which portions of the two processes are integrated is possible. For example, for the training process of data preprocessing, a data preprocessing model 92 may be configured with only the noise removal process for preprocessing a measurement noise in the physiological measurement data 82, and then the subjective score estimation model 93 may be configured to perform the feature extraction process, the dimensionality reduction process, and the subjective score estimation process end-to-end. In this case, it may be configured in the form of a deep learning model formed of a fully connected layer such as Long Short Term Memory, Graph Neural Network, Convolutional Neural Network, Graph Neural Network, Self Attention, and the like, which are excellent for handling time series data.

In addition, regarding the subjective score ground-truth data 91 to be estimated by the subjective score estimation model 93, when the subjective score ground-truth data 91 is obtained in daily life in particular, this often includes data subjectively annotated by the user himself or herself and therefore the ground-truth data itself may be unreliable. Considering that the subjective score estimation model 93 is trained with such a low-reliability ground-truth label, a model configuration suitable for this may be adopted. For example, the subjective score ground-truth data 91 may be weighted and trained according to the reliability of each answer of the user. In addition, a known algorithm may be used as a model configuration for ground-truth labels with low reliability and certainty. For example, based on the fact that the ground-truth labels are assigned to the entirety of the physiological measurement data 82 for the last 30 minutes, but that it is not strictly determined at what point in time the ground-truth label is made, the Multiple Instance Learning may be used. A Bayesian deep learning model or Label Distribution Learning may be used when a single ground-truth label cannot be determined but the reliability of the distribution position and order itself is relatively high.

Furthermore, likewise the data preprocessing model 92, the subjective score estimation model 93 may also have a model configuration that considers individual variabilities. It is considered that the subjective score ground-truth data 91, which is the ground-truth label learned by the subjective score estimation model 93, includes differences in cognition, such as the subjective cognition method for a certain event and the answer tendencies that occur when the recognized content is verbalized. Therefore, when correction is performed for the physical variability of the individual variabilities in the data preprocessing model 92, the model may be configured to include a cognitive variability correction process for correcting such a cognitive variability even for the ground-truth label. In this case, it is possible to perform subjective score estimation that matches the cognitive tendency of the user, thereby obtaining the effect of improving suitability for the user and acceptability of the subjective score estimation result from the user.

For the cognitive variability correction process, a dedicated process for the subjective score estimation model 93 may be added, or the subjective score estimation model 93 itself may be configured as end-to-end model. For example, when the dedicated process is added, the response style of the user for the subjective score ground-truth data 91 may be regarded as the cognitive tendency of the user, and the subjective score estimation that applies to all users using a classification model or a regression model may be performed, and then a process of correcting the estimation result in consideration of the mid-point response style and the extreme response style, which are response styles, may be additionally performed to obtain the subjective score estimation data 84. Further, for the end-to-end configuration, a multi-task learning may be configured by constructing the subjective score estimation model 93 with a neural network, and dividing a layer close to the final layer for each user to divide for each user, thereby realizing estimation by learning the cognitive tendency according to the user, or the subjective score estimation model 93 that is generic to all users may be created, and may be subjected to fine-tuning for each user to suit for a specific user.

When the training process of the subjective score estimation model 93 is completed as described above, the subjective score assessment unit 53 generates the subjective score estimation data 84 from the preprocessed data 83 by using the trained subjective score estimation model 93.

In the training process of the deviation assessment model S34, the deviation assessment unit 54 trains the deviation assessment model 94 with the physiological measurement data 82 and the preprocessed data 83. The deviation assessment model 94 assesses, as a deviation, the extent that the input physiological measurement data 82 deviates from normal state. The deviation assessment model 94 is typically an unsupervised model based on a known anomaly detection algorithm, and can be configured as a known statistical model or machine learning model. As the statistical model, for example, a statistical model that outputs a z-score using the average value and standard deviation for the data distribution of the preprocessed data 83 obtained by the data preprocessing model 92, a statistical model that outputs deviation values, a statistical model that outputs quantile points on data distribution, and the like may be used. As the machine learning model, for example, a machine learning model that outputs the distance based on the cluster center of the data distribution estimated non-parametrically from the preprocessed data 83 may be used.

For example, when the data preprocessing model 92 is hierarchically configured as a physical variability correction model to remove individual variabilities due to physical characteristics between individuals, and a feature extraction model such as Variational Auto Encoder or the like that may be used commonly among multiple users with individual variabilities due to the physical characteristics between individuals removed, it is possible to calculate inter-individual common estimated physiological measurement data which is obtained by inputting the preprocessed data 83 to the decoder unit of the feature extraction model, and estimated physiological measurement data for each individual, which is obtained from the inter-individual common estimated physiological measurement data by inverse transformation using the physical variability correction model. In this case, the deviation assessment model 94 may be trained as a model for calculating a reconstruction error that indicates the extent that the common estimated physiological measurement data between individuals can be reconstructed from the physiological measurement data removed of the individual variabilities due to the physical characteristics between individuals. In addition, the deviation assessment model 94 may be trained as a model for calculating a reconstruction error that indicates the extent that the estimated physiological measurement data for each individual can be reconstructed from the physiological measurement data 82. Furthermore, the deviation assessment model 94 may also be configured as a model for assessing the reconstruction errors of the two processes together and calculating the weighted values of the reconstruction errors. In the configuration described above, the deviation is smaller as closer to the physiological measurement data 82 measured in normal times, and the deviation is larger when there is an anomaly in the physiological state or when there is a problem with the measurement conditions of the physiological state, such that the difference between the input physiological measurement data 82 itself and the normal state may be assessed as the deviation.

When the training process of the deviation assessment model 94 is completed as described above, by using the trained deviation assessment model 94, the deviation assessment unit 54 generates the deviation data 85 from the physiological measurement data 82 and the preprocessed data 83.

In the training process of the uncertainty assessment model S35, the uncertainty assessment unit 55 trains the uncertainty assessment model 95 with the preprocessed data 83 and the subjective score estimation data 84. Typically, the subjective score estimation model 93 may be used as the uncertainty assessment model 95. In this case, by adding a perturbation to the preprocessed data 83 that was input to the subjective score estimation model 93 and used to calculate the subjective score estimation data 84, it is possible to assess, as an uncertainty, the extent that the subjective score estimation data 84 is varied, and use it as the uncertainty assessment model 95. In addition, when the subjective score estimation model 93 is configured by a Bayesian deep learning model or Label Distribution Learning that takes the uncertainty of the input data and the model into consideration, since the subjective score estimation data 84 is obtained as a distribution having a spread indicating the degree of certainty of estimation, the uncertainty assessment model 95 may be trained as a model for assessing the spread of the subjective score estimation data 84. As described above, by calculating the subjective score estimation data 84 from the physiological measurement data 82, apart from the degree of anomaly of the physiological measurement data 82, it is possible to assess the extent that the subjective state itself corresponding to the estimated input is uncertain and how different it is from the normal state.

When the training process of the uncertainty assessment model 95 is completed as described above, by using the trained uncertainty assessment model 95, the uncertainty assessment unit 55 generates uncertainty data 86 from the preprocessed data 83 and the subjective score estimation data 84.

In the training process of the discrepancy assessment model S36, the discrepancy assessment unit 56 trains the discrepancy assessment model 96 with the deviation data 85 and the uncertainty data 86. The discrepancy assessment model 96 is typically an unsupervised model based on a known anomaly detection algorithm that assesses anomalies using the deviation data 85 and the uncertainty data 86 as inputs, and may be configured as a known statistical model or machine learning model.

Further, instead of being based on learning, the discrepancy assessment model 96 may be configured to assess a belief about the extent that the deviation data 85 and the uncertainty data 86 are weighted as the discrepancy. For example, when emphasizing the difference between the input physiological measurement data 82 itself and the normal state, the deviation data 85 may be multiplied by 0.9 as a coefficient A, the uncertainty data 86 may be multiplied by 0.1 as a coefficient B, and the weighted sum thereof may be calculated to assess the discrepancy. In addition, when emphasizing the extent that the subjective state itself corresponding to the estimated input is uncertain, the deviation data 85 may be multiplied by 0.1 as a coefficient A, the uncertainty data 86 may be multiplied by 0.9 as a coefficient B, and the weighted sum thereof may be calculated to assess the discrepancy. In this case, the discrepancy is calculated as coefficient A x deviation+coefficient B x uncertainty, and a set of coefficients A and B is stored as the discrepancy assessment model 96.

As a result, for the complicated relationship of the physiological state and the subjective state of the mind and body, by considering both the distance between the physiological state itself and the normal state, and the extent that the subjective state of the mind and body itself is uncertain and how different it is from usual, it is possible to calculate an index for determining the appropriate timing of implementing intervention measures and timing of reporting to collect the data for the subjective state estimation model.

When the training process of the discrepancy assessment model 96 is completed as described above, by using the trained discrepancy assessment model 96, the discrepancy assessment unit 56 generates the discrepancy data 87 from the deviation data 85 and the uncertainty data 86.

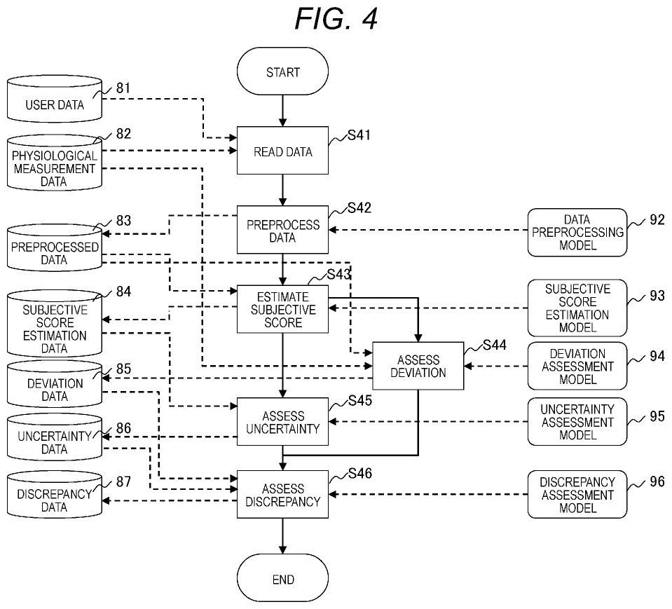

The series of training processes shown in FIG. 3 are executed at least once before the process of assessing the discrepancy regarding the subjective score estimation shown in FIG. 4 which will be described below. In addition, this process may be performed at regular intervals as the subjective score ground-truth data 91 and the physiological measurement data 82 are increased, and each of the data preprocessing model 92, the subjective score estimation model 93, the deviation assessment model 94, the uncertainty assessment model 95, and the discrepancy assessment model 96 may be re-trained. As described above, each model with higher assessment accuracy can be generated.

In addition, while it is described with reference to the example of using one model for each of the data preprocessing model 92, the subjective score estimation model 93, the deviation assessment model 94, the uncertainty assessment model 95, and the discrepancy assessment model 96, it is typically preferable to prepare multiple models for each and use them in combination. For example, for the discrepancy assessment model 96, multiple sets of coefficients A and B may be prepared such that multiple different discrepancies may be assessed, as the discrepancies, with respect to the belief of how much importance is placed on the deviation data 85 and the uncertainty data 86. As described above, when performing a report determination based on the discrepancy as will be described below, it is possible to implement a report determination from various viewpoints.

FIG. 4 is a flowchart provided to explain an example of a discrepancy assessment process in subjective score estimation performed by the physiological measurement data processing device 1. First, the reception unit 51 of the physiological measurement data processing device 1 performs a data read process S41 of reading the physiological measurement data 82, and the user data 81 storing the information of the user from whom the physiological measurement data 82 is acquired.

Then, the preprocessing unit 52 reads out the data preprocessing model 92, and performs data preprocessing S42 on the user data 81 and the physiological measurement data 82 using the read data preprocessing model 92, and outputs the preprocessed data 83.

In a subjective score estimation process S43, the subjective score assessment unit 53 reads out the subjective score estimation model 93 and generates the subjective score estimation data 84 from the read subjective score estimation model 93 and the preprocessed data 83. In a deviation assessment process S44, the deviation assessment unit 54 reads the deviation assessment model 94, and generates the deviation data 85 from the read deviation assessment model 94, the physiological measurement data 82, and the preprocessed data 83. In addition, in an uncertainty assessment process S45, the uncertainty assessment unit 55 reads the uncertainty assessment model 95, and generates the uncertainty data 86 from the read uncertainty assessment model 95, the preprocessed data 83, and the subjective score estimation data 84. While it is described as an example that the subjective score estimation process S43, the uncertainty assessment process S45, and the deviation assessment process S44 are performed in parallel, the aspects are not necessarily limited to this configuration. For example, the subjective score estimation process S43, the deviation assessment process S44, and the uncertainty assessment process S45 may be performed sequentially. In a discrepancy assessment process S46, the discrepancy assessment unit 56 reads the discrepancy assessment model 96, and generates the discrepancy data 87 from the read discrepancy assessment model 96, the deviation data 85, and the uncertainty data 86.

As described above, based on the deviation data 85 indicating the deviation of the physiological measurement data 82 itself from normal state and the uncertainty data 86 indicating the uncertainty of the subjective score estimation data 84 estimated based on the physiological measurement data 82, it is possible to generate the discrepancy data 87 which is the discrepancy indicating the state of the user. As a result, as an advantageous effect, it is possible to obtain a scale for determining the subjective state as to whether or not the user is in a subjective state different from usual.

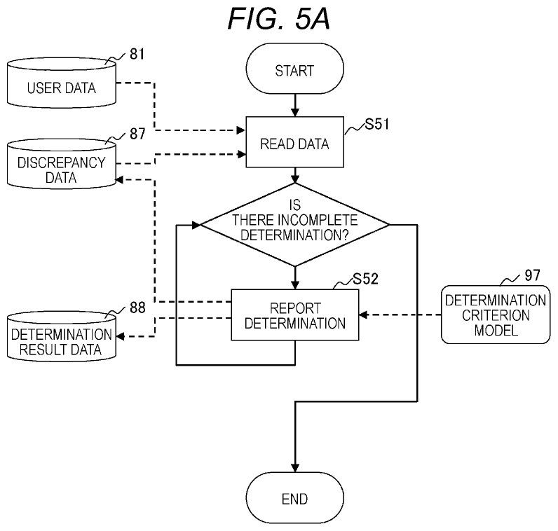

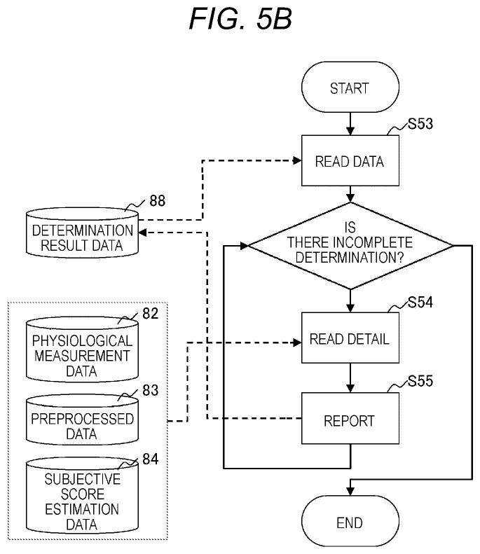

FIG. 5A is a flowchart provided to explain an example of a report determination process based on a discrepancy, which is performed by the report determination unit 57 of the physiological measurement data processing device 1. In the data read process S51, the report determination unit 57 reads the user data 81 and the discrepancy data 87. Then, a report determination process S52 is performed as long as there remains the discrepancy data 87 whose determination completion flag indicates incomplete.

In the report determination process S52, report determination is performed based on the determination criterion model 97 with respect to the discrepancy data 87 for which the determination completion flag included therein indicates incomplete, and the determination result is output to the determination result data 88. In addition, for discrepancy data 87 for which report determination is completed, the determination completion flag is updated to completed.

While it is described as an example that one type of determination criterion model 97 is used, aspects are not limited thereto, and multiple determination criterion models 97 may be prepared. For example, by preparing a plurality of determination criterion models 97 suitable for the discrepancy assessment model 96 for the discrepancy data 87 calculated by different discrepancy assessment models 96, an appropriate report determination process can be performed in consideration of the discrepancy calculation criteria. In addition, a plurality of determination criterion models 97 may be prepared for the discrepancy data 87 calculated from the single discrepancy assessment model 96. For example, by providing a first determination criterion model for determining whether or not a report criterion for reporting the user himself or herself is exceeded and providing a second determination criterion model for determining whether or not a report criterion for reporting to the administrator who manages the users is exceeded, it is possible to perform report determination based on different criteria depending on the recipient of the report, and determine which status is reported according to the action to be taken by the recipient of the report. Furthermore, if a plurality of determination criterion models 97 are used separately according to the characteristics of the user data 81, it is possible to perform report determination in consideration of information such as age and the like which is not included in the physiological measurement data 82 and the subjective score estimation data 84.

FIG. 5B is a flowchart provided to explain an example of a process of outputting a report based on the determination result data 88, which is performed by the report output unit 58 of the physiological measurement data processing device 1. In a data read process S53, the report output unit 58 reads the determination result data 88. Then, when there is unreported data having the report status as incomplete in the determination result data 88, the process of reporting to the recipient is performed.

First, when there is unreported data, details of the data related to the unreported determination result data 88 are read in a detail read process S54. In an example, the physiological measurement data 82, the preprocessed data 83, and the subjective score estimation data 84 are read in the detail reading process S54. By reading these related data, it is possible to report to the recipient by considering the information of the related data.

Then, a reporting process S55 is performed for the unreported data of the determination result data 88. In the reporting process S55, the recipient of the report is reported by the input and output device 5 via the network 9. For example, if the recipient of the report is the user, the reporting process S55 is performed for the user terminal 7 corresponding to the user ID of the determination result data 88, and the reporting device 15 of the user terminal 7 issues a report to the user. At this time, if a smartphone is used as the user terminal 7, the report may be given to the user in the form of a push report on the smartphone, a message on the report application, or the like. In addition, if the recipient of the report is the administrator, the reporting process S55 is performed for the administrator terminal 8 corresponding to the administrator who manages the user corresponding to the user ID of the determination result data 88, and the reporting device 33 of the administrator terminal 8 issues a report to the administrator. At this time, when a PC is used as the administrator terminal 8, the report may be given in the form of an alarm sound output through the PC, an e-mail sent to the PC, a report displayed on an administrator application, and the like. In addition, for the determination result data 88 for which the reporting process is completed, the report status flag is updated to completed.

As described above, it is possible to send a report to the recipient at an appropriate timing for intervention measures or data collection based on the subjective state, by considering whether or not the user is in a subjective state different from usual based on the discrepancy.

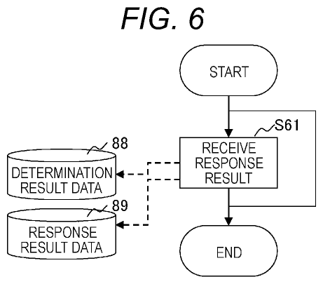

FIG. 6 is a flowchart provided to explain an example of a process of receiving a response result sent in response to a report output, which is performed by the physiological measurement data processing device 1. The reception unit 51 performs a response result receiving process S61 for receiving a reply from the terminal that received the report. In the following, as an example of receiving a response from the user terminal 7, it is assumed that the timing at which the user is considered to be in an emotional subjective state different from the normal state is reported to the user based on the discrepancy. In this case, in the response result receiving process S61, the ground-truth data representing the subjective state of emotion, which is generated based on the response of the user to the user terminal 7, is received as the response result data 89. In addition, when the response result data 89 is received, the corresponding status flag of the determination result data 88 is updated to completed.

As described above, in response to the report implemented at the appropriate timing for intervention measures and data collection, such as the timing when the user is in a subjective state different from usual, the result of action taken by the recipient who received the report can be obtained. For example, in an exemplary embodiment, in response to a report related to the timing of determining the subjective state of different emotion, by acquiring the subjective score ground-truth data 91 as the corresponding ground-truth data of the emotion at that moment, it is possible to efficiently acquire the ground-truth data of the emotions that rarely occur in daily life. Further, in the second embodiment described below, in response to a report related to the timing of determining the user to be in physical and mental health conditions different from usual, such as fatigue and mood, by acquiring a flag value indicating whether or not the user checks the report, the content of actions taken, and the actual subjective physical and mental health condition at that time, it is possible to acquire the response result data 89 for use in accuracy verification of subjective score estimation and intervention effect assessment.

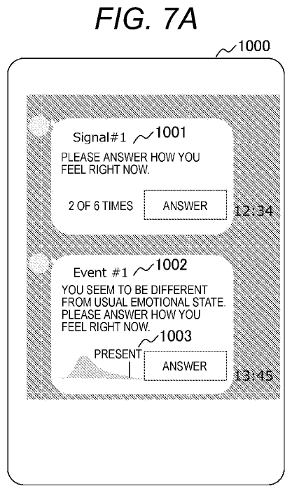

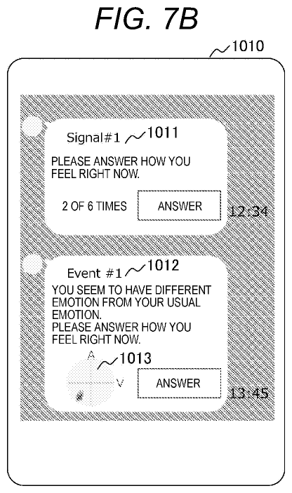

FIGS. 7A and 7B are diagrams showing examples of report output screens output by the result display unit 59 to the display of the input and output device 13 of the user terminal 7. In this example, it is described as an example that the reporting process is carried out in the form of a message sent to the user on a report application.

A display screen 1000 shown in FIG. 7A is an example of a display screen based on a discrepancy threshold. In an example where the present invention is not implemented, for example, the report application reports to the user irregularly (experience sampling based on time information) like Signal #1 1001, and attempts to collect the ground-truth data corresponding to an emotion at a certain moment. Since emotions such as strong happiness and sadness do not occur frequently in daily life, the way of simple irregular reporting takes a long time until the ground-truth data is collected, or a predetermined period ends without acquiring a sufficient number of ground-truth data.

On the other hand, if the discrepancy according to the embodiment is used, the timing at which the user is in a subjective state (emotional state) different from usual can be reported to the user terminal 7 in a form such as Event #1 1002 to prompt the user to input the emotional state. If the response based on this report is received as the response result data 89 in the receiving process of the response result, the effect of being able to acquire the subjective score ground-truth data 91 corresponding to a rare emotional state is obtained with high efficiency compared to acquiring the subjective score ground-truth data 91 which is emotional ground-truth data based on irregular reports such as Signal #1 1001. In addition, at this time, by providing a report that additionally displays a discrepancy 1003 at the time of report in comparison with the usual discrepancy, when generating the response result data 89, it is possible to obtain the effect that the user may obtain material for introspection of the content of the report.

A display screen 1010 shown in FIG. 7B is an example of a display screen that displays the subjective score estimation results when the subjective score estimation model 93 is created for each user. In this case, instead of presenting the abstract information like the discrepancy to the user, the example displays, as possible ranges, the extent that the emotional state of the subjective score estimated based on the physiological measurement data 82 is discrepant from the user himself or herself during normal times and then displays it as Event #1 1012. For example, the subjective state at that time may be shown with a distribution range on a two-dimensional graph 1013 of arousal and valence based on Russell's cyclic model.

As described above, the user feeds back the response result data 89 with reference to the information about how the user felt in the state at a certain moment, such that the physiological measurement data processing device 1 can collect the subjective score ground-truth data 91.

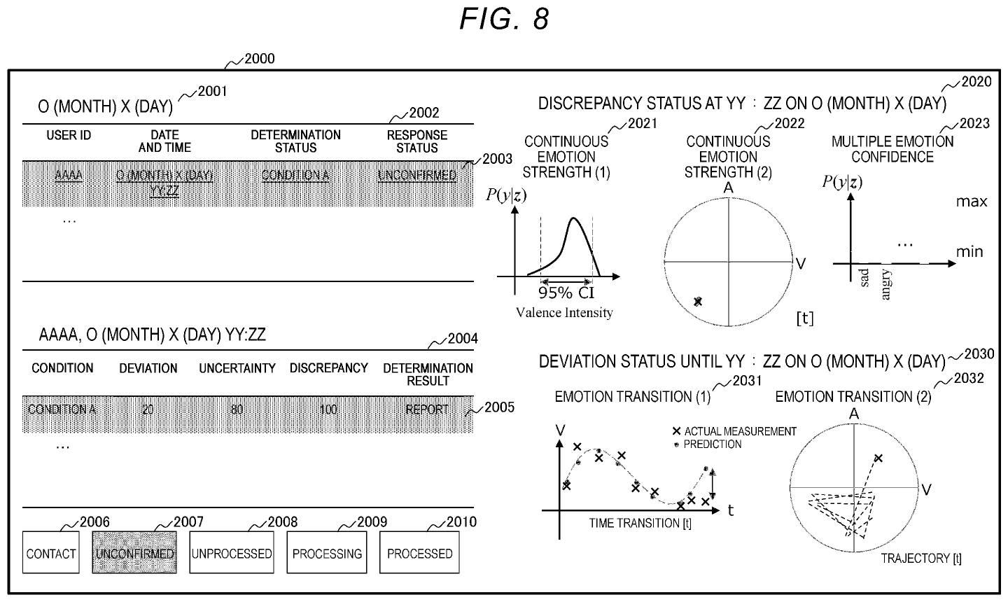

FIG. 8 is a diagram showing an example of a report output screen output by the result display unit 59 to the display of the input and output device of the administrator terminal 8. In this example, an example of a dashboard 2000 showing a list of report statuses for a series of group of users managed by the administrator is shown.

The dashboard 2000 is a display screen for listing and analyzing reports sent to a group of users on a certain date 2001. A table 2002 on the upper left side displays a list of reports and response statuses of the user to the reports. When a specific report 2003 is selected from this table, a plurality of discrepancy determination results corresponding to the subjective score estimation data 84 for reporting are displayed in a table 2004 on the lower left side. When selecting a row 2005 having the determination result indicating “report”, a contact button 2006 for contacting the user who is a recipient of the report in relation to the data reported, and a button for displaying the candidate actions performed or to be performed by the administrator in response to the determination result are displayed. Specifically, an unconfirmed button 2007 indicating that the content is not confirmed, an unprocessed button 2008 indicating that it is confirmed but the user has not taken any action, a processing button 2009 indicating that the user is taking an action, a processed button 2010 indicating that the user completed the response, and the like can be displayed. The buttons 2007 to 2010 display the current status, and the administrator can press the corresponding button each time he or she takes an action to update the status.

The right side of the dashboard 2000 displays details of the data corresponding to the row 2005 selected in the table 2004 on the lower left side.

In an upper right area 2020, the deviation status of the subjective score estimation at a certain point in time corresponding to the selected row 2005 is indicated by a plurality of means. For example, the first continuous emotion strength 2021 indicates a one-dimensional confidence interval when the interval estimation of arousal and valence is performed in the subjective score estimation. The second continuous emotion strength 2022 is an example of displaying a similar confidence interval as a two-dimensional graph. The multiple emotion confidence 2023 is an example of displaying the degree of each basic emotion as a probability value when a plurality of basic emotions are estimated in addition to the arousal and valence. Even in this case, the administrator may confirm the degree of emotional variation and the uncertainty of the estimation from the minimum and maximum values of the estimated probability of each basic emotion. As described above, the administrator is able to visually determine the extent that the subjective state of the user is discrepant from the normal states at a certain point in time.

A lower right area 2030 is an example of showing, by a plurality of means, the deviation status of the subjective score estimation corresponding to the selected row 2005, taking into consideration the estimation process on the same measurement date before the certain time. For example, in a first emotion transition 2031, the time-series change of the one-dimensional emotion estimation value in the first continuous emotion strength 2021 and the originally predicted subjective estimation data are one-dimensionally displayed. Furthermore, in a second emotion transition 2032, the trajectory of the transitioning of the subjective score estimation data 84 in the second continuous emotion strength 2022 is shown on the two-dimensional graph. As described above, it is possible to visually check the discrepancy from the normal state, taking into account the state during the day.

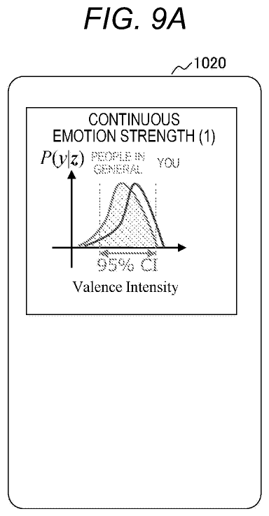

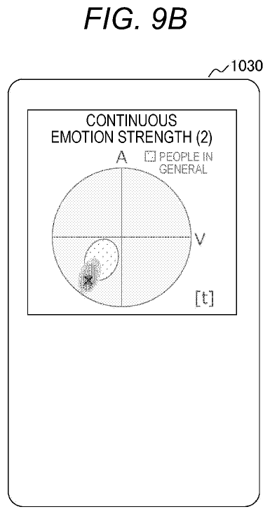

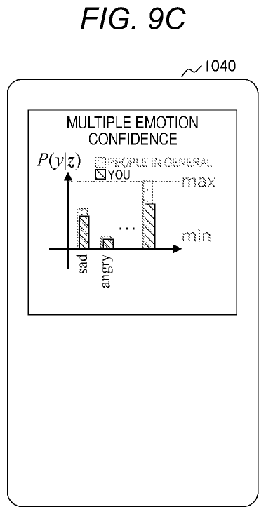

FIGS. 9A to 9C are diagrams showing examples of screens displaying subjective score estimation results displayed on the user terminal 7. Like the example of FIG. 8 showing the dashboard for the administrator, it is possible to display the same content (e.g., the first continuous emotion strength 2021, the second continuous emotion strength 2022, the multiple emotion confidence 2023) as the discrepancy status in the subjective score estimation at a certain point in time for the user as well. In particular, when a plurality of subjective score estimation models 93 are prepared for each user, and the like, by using the subjective score estimation data 84 estimated based on the subjective score estimation model 93 for the user himself or herself and the subjective score estimation data 84 estimated by the subjective score estimation model 93 of a plurality of users other than the user himself or herself, it is easy to understand how much the situation of the subjective score estimation data 84 at a certain point in time is different from that of other users in general. For example, a display screen 1020 (FIG. 9A) shows the first continuous emotion strength, a display screen 1030 (FIG. 9B) shows the second continuous emotion strength, and a display screen 1040 (FIG. 9C) shows the multiple emotion confidence such that the subjective score estimation data of the user and the subjective score estimation data of general others can be compared. As a result, it is possible for the user to understand that the estimation result is suitable for the user himself or herself, and improved acceptability of the estimation content of the subjective score estimation data 84 is expected.

Next, the characteristic structure of each data used in the physiological measurement data processing device 1 will be described.

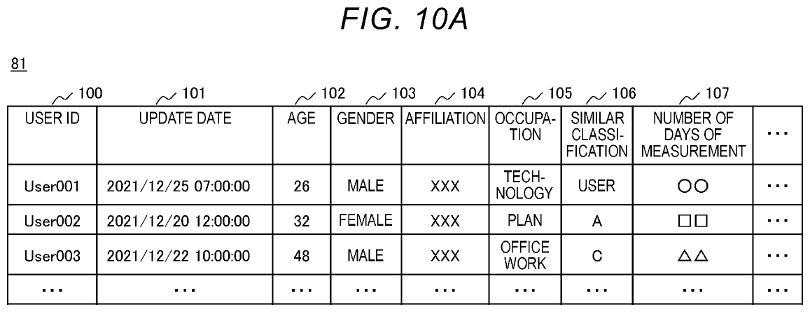

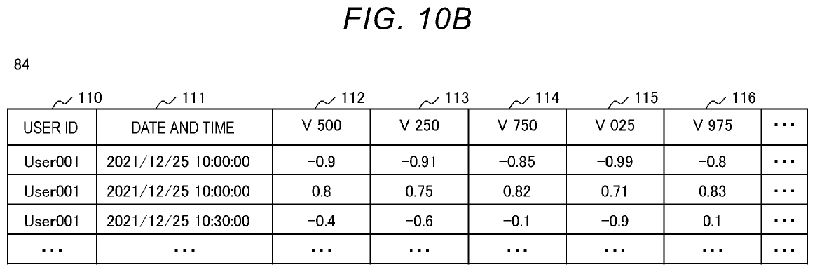

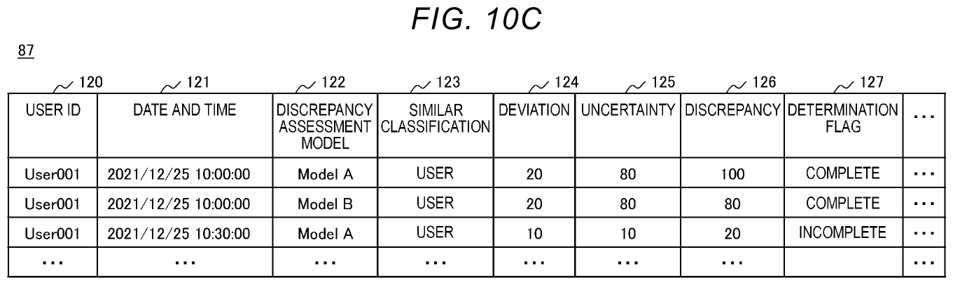

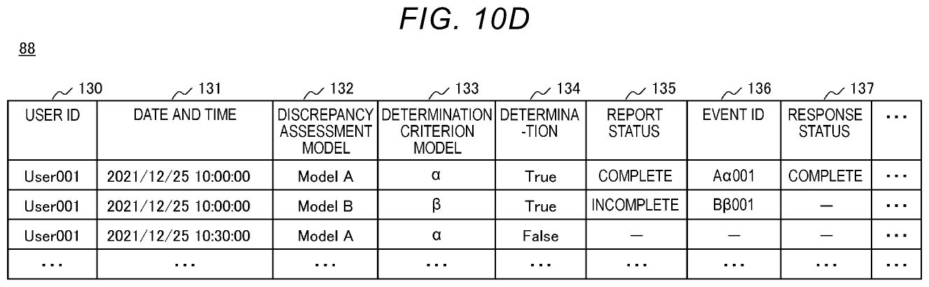

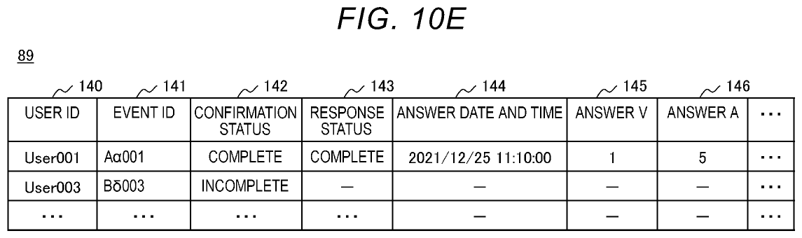

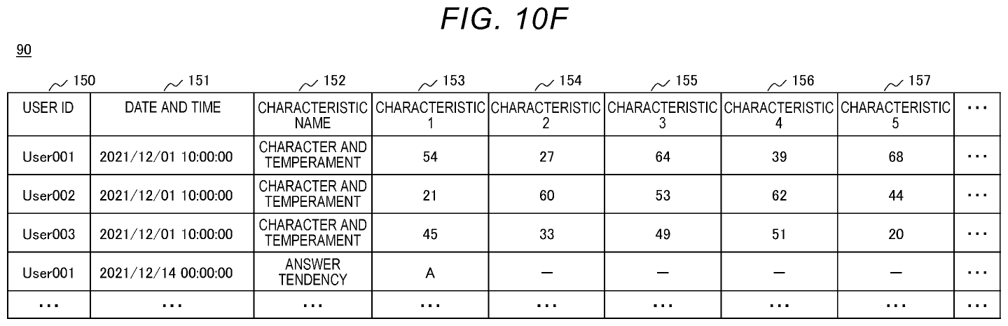

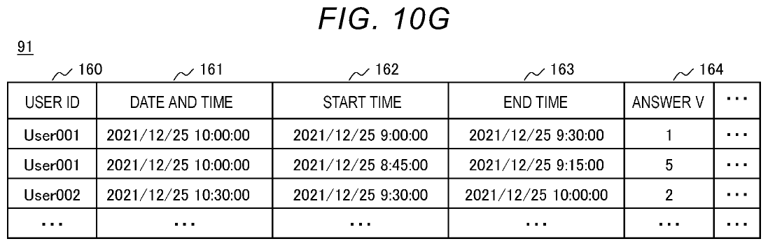

FIG. 10A is a diagram showing an example of the data structure of the user data 81 held in the physiological measurement data processing device 1. The user data 81 typically stores a user ID 100, an update date 101, an age 102, a gender 103, an affiliation 104, an occupation 105, and the like. In addition, for example, when performing a similar user classification process to be described below as the third embodiment, a similar classification 106 indicating a similar user category and a number-of-days 107 counted so far serving as an index for determining whether or not to apply the similar user classification process may be stored.

FIG. 10B is a diagram showing an example of the data structure of the subjective score estimation data 84 held in the physiological measurement data processing device 1. The subjective score estimation data 84 typically stores a user ID 110, a date and time 111, and an estimated subjective score. For example, the subjective score estimation data 84 corresponding to the first continuous emotion strength in FIG. 9A may store the quantiles of values obtained by normalizing the strength value of valence from −1 to 1. In this case, the median valence estimate is stored in V_500 112, and 25%, 75%, 2.5%, and 97.5% quantiles are stored in V_250 113, V_750 114, V_025 115, and V_975 116 respectively. Note that when subjective scores are estimated using a plurality of subjective score estimation models, even if the same data is used, the estimated subjective scores may differ (the first and second rows in FIG. 10B).

FIG. 10C is a diagram showing an example of the data structure of the discrepancy data 87 held in the physiological measurement data processing device 1. The discrepancy data 87 typically stores a user ID 120, a date and time 121, a discrepancy assessment model 122 indicating model information used to calculate the discrepancy, a deviation 124, an uncertainty 125, a discrepancy 126, a determination completion flag 127 that stores a determination completion flag, and the like. In addition, for example, when the similar user classification process to be described below as the third embodiment is performed, a similar classification 123 may be stored that indicates whether or not the similar user classification is used in calculating the discrepancy.

FIG. 10D is a diagram showing an example of the data structure of the determination result data 88 held in the physiological measurement data processing device 1. The determination result data 88 typically stores a user ID 130, a date and time 131, a discrepancy assessment model 132, a determination criterion model 133 used as a criterion for determining whether or not to report, a determination 134 that stores the determination result, a report status 135, an event ID 136 for uniquely specifying the determination result report event, and a corresponding status 137 for the report.

FIG. 10E is a diagram showing an example of the data structure of the response result data 89 held in the physiological measurement data processing device 1. Typically, the response result data 89 stores a user ID 140, an event ID 141, a report confirmation status 142, a report corresponding status 143, an answer date and time 144, and the like. Further, when collecting ground-truth data regarding emotions, an answer V 145 storing the ground-truth data of the valence, and an answer A 146 storing the ground-truth data of the arousal may be stored as the responses from the user.

FIG. 10F is a diagram showing an example of the data structure of the user characteristic data 90 held in the physiological measurement data processing device 1. The user characteristic data 90 is used in the third embodiment which will be described below. The user characteristic data 90 typically stores a user ID 150, a date and time 151, and a characteristic name 152. In addition, when the Big-Five Personality is measured by NEO-PI-R or NEO-FFI as the characteristics, the scores of five types of characteristics corresponding to the Big-Five personality may be stored in characteristic 1 153 to characteristic 5 157.

FIG. 10G is a diagram showing an example of the data structure of the subjective score ground-truth data 91 held in the physiological measurement data processing device 1. The subjective score ground-truth data 91 typically stores a user ID 160, a date and time 161 indicating the date and time when the user answered, a start time 162 and an end time 163 of the period when the user determined the subjective state of mind and body, and the like. In addition, when collecting ground-truth data regarding emotions, an answer V 164 storing ground-truth data of valence may be stored as a response from the user.

As described above, the physiological measurement data processing device 1 of the embodiment includes the reception unit 51 that receives the physiological measurement data 82 of the user, the deviation assessment unit 54 that assesses the deviation from the past physiological measurement data 82 based on the physiological measurement data 82, the uncertainty assessment unit 55 that assesses the uncertainty when estimating a subjective score indicating the subjectivity of the user based on the physiological measurement data 82, and the discrepancy assessment unit 56 that assesses the discrepancy indicating the discrepancy from the usual state of the user based on the deviation and the uncertainty. Further, the report determination unit 57 performs report determination, and when the discrepancy data 87 exceeds the discrepancy threshold value, outputs a report to the recipient.

As a result, since the physiological measurement data processing device 1 of the embodiment assesses and outputs the discrepancy indicating the state of the user based on the uncertainty of the subjective score estimated based on the physiological measurement data 82 in addition to the deviation of the physiological measurement data 82 itself, there is an effect that it is possible to use the discrepancy to take actions such as reporting for intervention measures, collection of ground-truth data, and the like while considering the subjective state.

### Second Embodiment

Except for the differences described below, each component of the physiological measurement data processing device 1 of the second embodiment has the same function as those denoted by the same reference numerals in the first embodiment, and redundant description will be omitted.

The second embodiment is an example of having the subjective mood regarding user's own health, such as QoL and wellbeing in daily life as an estimation target for the subjective state of mind and body to be estimated, and implementing an intervention measure at an appropriate timing when the user is in a subjective state different from usual or is estimated to be in a different subjective state in the future. If the natural emotions in daily life estimated in the first embodiment are taken as the subjective state of mind and body at a certain point in time, the subjective mood estimated in the second embodiment may be said to be a higher-order subjective state of mind and body that appears as an accumulation or variation of these. Therefore, in the second embodiment, the subjective score estimation model 93 has a multistage configuration of a first subjective score estimation sub-model and a second subjective score estimation sub-model, the first subjective score estimation data as a latent variable is obtained from the physiological measurement data 82 by the first subjective score estimation sub-model, the final second subjective score estimation data is obtained using the first subjective score estimation data, which is a latent variable, by the second subjective score estimation sub-model, and the finally obtained second subjective score estimation data is used as subjective score estimation data 84, and the report determination is performed based on the discrepancy. The second subjective score estimation data may be estimated by a plurality of types of first subjective score estimation data estimated by a plurality of first subjective score estimation sub-models. As described above, in the second embodiment, by configuring the subjective score estimation model 93 in multiple stages, it is possible to estimate a higher-order subjective state of mind and body and act on the user based on the determination result.

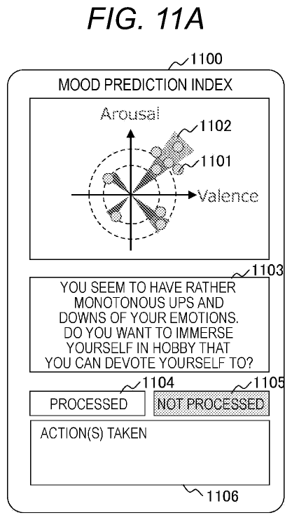

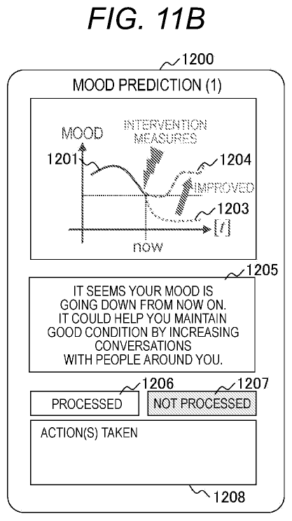

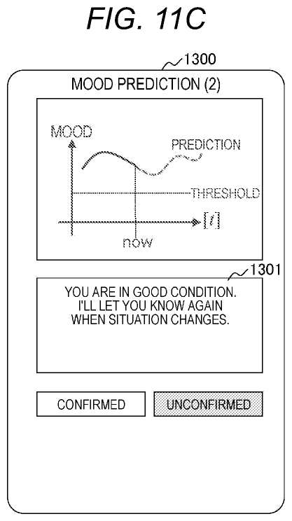

FIGS. 11A to 11C are diagrams showing examples of screens displaying the subjective score estimation result display screen displayed on the user terminal 7. In this embodiment, when it is estimated or predicted that the subjective state will be different from usual in the future, such as when a depressed mood is predicted, an appropriate report for intervention based on the prediction is sent to the user. An example of the subjective score estimation process will be described below, in which the physiological measurement data 82 is time-divided, the emotional state is estimated from each of the time-divided physiological measurement data 82, and the mood is estimated from variations in the emotional state.

For example, a mood prediction index display screen 1100 shown in FIG. 11A is an example of showing the user the grounds for mood estimation when natural emotions in daily life are used as explanatory variables for future mood estimation (first subjective score estimation data). The natural emotions in daily life may be estimated by the first subjective score estimation sub-model, like the subjective score estimation model shown in the first embodiment. In this example, first, each emotional state estimated from each time-divided physiological measurement data 82 is plotted on a two-dimensional graph by points 1101. The frequency of appearance of the estimated emotional state in each quadrant is shown in three stages in a radar chart 1102 in such a way that it is superimposed on this two-dimensional plot. The appearance frequency distribution of this estimated emotional state represents the diversity of emotional experiences (emodiversity) in a certain time period, and the mood is estimated based on this appearance frequency distribution.

In this example, the point cloud and radar chart are biased toward the first quadrant, suggesting a low diversity of emotional experiences. When such a situation is detected, since the ups and downs of emotions seem to be monotonous, an example of measures for improving the situation is reported as an advice 1103. Furthermore, a processed button 1104 indicating that the user who received the report has taken a certain action on the report, or an unprocessed button 1105 indicating that the user has not taken any action may be provided, or a comment field 1106 for transmitting the details of the action to the physiological measurement data processing device 1 as the response result data 89 when action is performed may be provided.

A first mood prediction display screen 1200 shown in FIG. 11B is an example of a display screen for estimating the future mood of the user estimated using the subjective score estimation data and reporting to the user when the depression is predicted. In this case, in addition to the estimated series 1201 of mood transitions so far, a future change prediction 1203 and a mood change prediction 1204 when a certain intervention measure is taken are displayed. In addition, an advice 1205 to be implemented in order to prevent depression is given to the user in a report. Furthermore, a processed button 1206 indicating that the user who received the report has taken a certain action on the report, or an unprocessed button 1207 indicating that the user has not taken any action may be provided, or a comment field 1208 for transmitting the details of the action to the physiological measurement data processing device 1 as the response result data 89 when action is performed may be provided.

A second mood prediction display screen 1300 shown in FIG. 11C shows an example of a display screen displayed to the user when the future mood of the user estimated using the subjective score estimation data is estimated to be a normal change. Even when no deviation from the normal state is detected, the user may check the estimation result on the screen to confirm the estimation status of the present and future subjective states. In this case, since a good condition is maintained as usual, the comment column 1301 displays that the status quo is recommended.

As described above, the physiological measurement data processing device 1 of the present embodiment may have an effect of being able to take actions such as intervention measures, reports for ground-truth data collection, and the like while considering the subjective state, when changes in the final subjective state differ from normal state due to changes in one or more subjective states.

### Third Embodiment

Except for the differences to be described below, each component of the physiological measurement data processing device 1 of a third embodiment has the same function as those denoted by the same reference numerals in the first embodiment or the second embodiment, and redundant description will be omitted.

In the first and second embodiments, each model used in the data preprocessing S42, the subjective score estimation process S43, the deviation assessment process S44, the uncertainty assessment process S45, and the discrepancy assessment process S46 uses a user-specific model trained with the data of the user himself or herself, or a model that may be used commonly among multiple users is trained after removing individual variabilities for each user in the data preprocessing S42 and the subjective score estimation process S43. Meanwhile, if the model for each user is retrained each time a new user is added, the data collection cost for new users increases, and it takes a great deal of time to collect a sufficient amount of physiological measurement data 82 for training, resulting in opportunity loss. In the third embodiment, similar user classification process is performed in order to enable new users to use the system at an early stage.

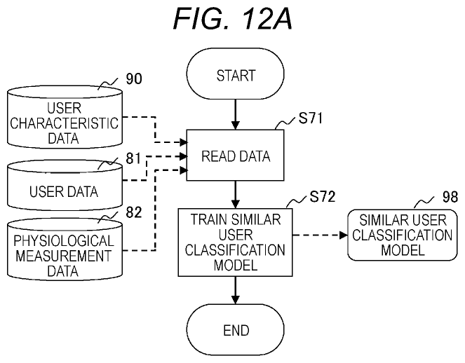

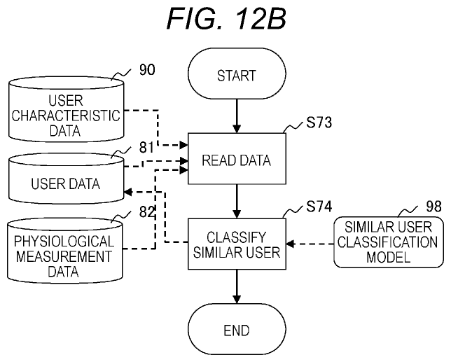

FIG. 12A is a flowchart showing an example of training process for similar user classification performed by the similar user classification unit 60 of the physiological measurement data processing device 1. First, in a data read process S71, the similar user classification unit 60 reads the user characteristic data 90 (see FIG. 10F) and the user data 81 (see FIG. 10A). In addition, the physiological measurement data 82 may also be read. In addition, the physiological measurement data 82 read at this time may be a small amount of data compared to the amount required for re-training for the user of each model used in the data preprocessing S42, the subjective score estimation process S43, the deviation assessment process S44, the uncertainty assessment process S45, and the discrepancy assessment process S46. That is, even when the physiological measurement data 82 is read in the data read process S71, the large amount of physiological measurement data 82 required for re-training does not need to be measured in advance from new users, and the effect of reducing the measurement cost is obtained.

Then, the training process of the similar user classification model S72 is performed using the read data. In the training process of the similar user classification model S72, a similar user classification model 98 for classifying or characterizing subjects is generated based on information on users with low measurement cost. For example, it is known that the answer tendency of the subjective score ground-truth data 91 of the user is influenced by the character and temperament of the user himself or herself and the like. Therefore, as the user characteristic data 90 representing the characteristics of the user, for example, personality and temperament information based on Big Five Personality, occupational group information, answer tendency to psychological scales such as mid-point response style and extreme response style, amount level of motivation for intervention, and the like may be used. Further, user attribute information such as gender and age stored in the user data 81 may also be used. In addition, the average heart rate that can be characterized from a small number of physiological measurement data 82, the physiological measurement data 82 such as blood pressure information which is considered to be related to the awareness of health of the user and the like may be used.

A known algorithm may be used for training the similar user classification model 98. For example, as a machine learning algorithm that classifies data without supervision, a hard clustering algorithm using the k-means method or the spectral clustering method may be used to train the similar user classification model 98 that classifies users with similar characteristics into several groups of similar users. A soft clustering algorithm such as a Gaussian mixture model or a mixture Dirichlet process may also be used to train the similar user classification model 98 that estimates the degree of closeness to a representative group of users. Then, for each obtained group of similar users, each model used in the data preprocessing S42, the subjective score estimation process S43, the deviation assessment process S44, the uncertainty assessment process S45, and the discrepancy assessment process S46 is trained according to the flowchart provided to explain FIG. 3.

FIG. 12B is a flowchart provided to explain an example of similar user classification process performed by the similar user classification unit 60 of the physiological measurement data processing device 1. In a data read process S73, the similar user classification unit 60 reads the user characteristic data 90 (see FIG. 10F) and the user data 81 (see FIG. 10A). In addition, the physiological measurement data 82 may be read. Then, in a similar user classification process S74, the similar user classification model 98 is read, and the read data is used to classify new users into groups of similar users, and information on the groups of similar users is stored in the user data 81. Since each data used for the similar user classification process has a low measurement cost, the similar user classification process S74 may be executed early even for new users.

Then, by using each model used in the data preprocessing S42, the subjective score estimation process S43, the deviation assessment process S44, the uncertainty assessment process S45, and the discrepancy assessment process S46, which are trained for each group of similar users by using the obtained information on the groups of similar users, a series of assessment processes related to subjective score estimation may be performed according to the flowchart provided to explain FIG. 4.

As described above, even for a new user, without waiting for the necessary amount of physiological measurement data 82 to be accumulated for the user to re-train the entire model, it is possible to detect when the new user is in a subjective state different from usual at an early stage and report this at the appropriate time.

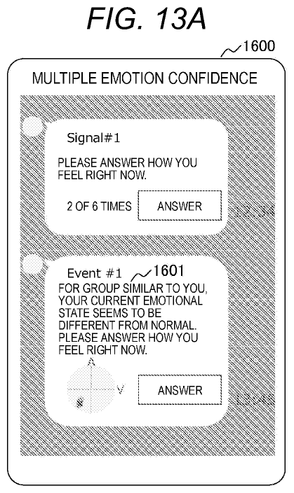

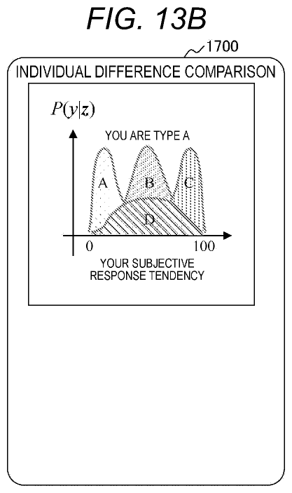

FIGS. 13A and 13B are diagrams showing examples of output screens displayed on the user terminal 7. A display screen 1600 showing the subjective score estimation result in FIG. 13A may report that the user is in a state different from the normal state of the group of similar users (Event #1 1601), instead of displaying, in the report message (Event #1 1012) on the display screen 1010 of FIG. 7B, that the user is in the subjective state different from usual state for the user himself or herself. As a result, functions may be provided to new users at an early stage, and the reported user can understand that the estimation may include errors because the results are not individualized only for the user himself or herself. Further, on a individual variability comparison display screen 1700 shown in FIG. 11B, it is possible to check the difference in characteristics for each group of similar users. For example, in the classification of group of similar users, if the subjective answer tendency is used as one index for classification, the difference in the subjective answer tendency for each group of similar users may also be displayed. As a result, the user can determine what their own characteristics are compared to other users and a group of similar users.

As described above, in the third embodiment, in order to make each model in the first and second embodiments available to the new users at an early stage, users are classified into groups of similar users by using the user characteristic data 90 with a low measurement cost or a shorter required period, and each model is trained for each group of similar users. As a result, it is possible to quickly take actions for new users, such as intervention measures and reports for ground-truth data collection, while considering their subjective states.

Note that the present invention is not limited to the embodiments described above, and includes various modifications. For example, the embodiments described above are described in detail in order to explain the present invention in an easy-to-understand manner, and are not necessarily limited to those having all the configurations described above. Further, a part of the configuration of an embodiment may be replaced with the configuration of another embodiment, and the configuration of another embodiment may be added to the configuration of an embodiment. In addition, it is possible to add, delete, and replace other configurations for a part of the configuration of each embodiment.

Each of the configurations, functions, processing units, processing means, and the like described above may be implemented by hardware by designing a part or all of those with, for example, an integrated circuit. Each of the configurations, functions, and the like described above may be implemented by software by interpreting and executing a program that realizes each function by the processor 2. The information such as a program, a table, a file, and the like that implements each function may be stored in a storage device such as the memory 3, a hard disk drive, a solid state drive (SSD), or a computer-readable non-transitory data storage medium such as an IC card, an SD card, DVD, and the like.

In addition, the drawings illustrate control lines and information lines that are considered necessary for explaining the embodiments, and do not necessarily show all the control lines and information lines included in the actual product to which the present invention is applied. In practice, it may be considered that almost all components are connected to each other.

What is claimed is:

1. A physiological measurement data processing device comprising:
a reception unit that receives, from a user terminal of a user, physiological measurement data obtained by measuring a physiological state of the user with a sensor;
a deviation assessment unit that assesses a deviation of the physiological measurement data received by the reception unit with respect to past physiological measurement data of the user;
a subjective score assessment unit that estimates a subjective score indicating a subjective state of mind and body of the user based on the physiological measurement data received by the reception unit;
an uncertainty assessment unit that assesses an uncertainty of the subjective score estimated by the subjective score assessment unit; and
a discrepancy assessment unit that assesses a discrepancy indicating a discrepancy with respect to a usual state of the user based on the deviation assessed by the deviation assessment unit and the uncertainty assessed by the uncertainty assessment unit.

2. The physiological measurement data processing device according to claim 1, further comprising a report determination unit that performs report determination based on the discrepancy assessed by the discrepancy assessment unit.

3. The physiological measurement data processing device according to claim 2, wherein the subjective score assessment unit estimates a subjective score of the user by using a subjective score estimation model, and
the subjective score estimation model is trained by using a set of subjective score ground-truth data obtained by the user inputting the subjective state of mind and body of the user into the user terminal, and preprocessed data obtained by subjecting the physiological measurement data of the user for a period corresponding to the subjective score ground-truth data to a predetermined preprocessing.

4. The physiological measurement data processing device according to claim 3, wherein, based on the discrepancy assessed by the discrepancy assessment unit, the report determination unit determines whether or not to issue a report prompting the user to input the subjective state of mind and body of the user to the user terminal of the user.

5. The physiological measurement data processing device according to claim 4, further comprising a result display unit for displaying, in the report, the discrepancy assessed by the discrepancy assessment unit or the subjective score estimated by the subjective score assessment unit on the user terminal of the user.

6. The physiological measurement data processing device according to claim 3, wherein the predetermined preprocessing includes a correction process for removing individual variabilities due to physical characteristic, and a process of extracting features from the physiological measurement data of the user that is subjected to the correction process, and
the subjective score estimation model is a model that inputs a feature value obtained by the process of extracting features.

7. The physiological measurement data processing device according to claim 6, wherein the subjective score estimation model is trained using the subjective score ground-truth data obtained by performing a cognitive variability correction process based on a cognitive variability of the user with respect to the subjective score ground-truth data which is the subjective state of mind and body of the user input by the user into the user terminal of the user.

8. The physiological measurement data processing device according to claim 3, wherein the subjective score estimation model includes a first subjective score estimation sub-model and a second subjective score estimation sub-model,
the first subjective score estimation sub-model estimates a first subjective score indicating the subjective state of mind and body of the user based on the physiological measurement data received by the reception unit,
the second subjective score estimation sub-model estimates a second subjective score indicating the subjective state of mind and body of the user based on the first subjective score, and
the subjective score assessment unit outputs the second subjective score as a subjective score to be estimated.

9. The physiological measurement data processing device according to claim 8, wherein the report determination unit determines whether or not to report the user terminal of the user of advice for improving the subjective state of mind and body, based on the discrepancy assessed by the discrepancy assessment unit.

10. The physiological measurement data processing device according to claim 3, further comprising a similar user classification unit that estimates a group of similar users similar to the user, wherein
the deviation assessment unit assesses a deviation of the physiological measurement data received by the reception unit with respect to past physiological measurement data of the estimated group of similar users who are similar to the user,
the subjective score estimation model is trained using a set of the subjective score ground-truth data and the preprocessed data of the group of similar users, and
the discrepancy assessment unit assesses a discrepancy indicating a discrepancy from a usual state of the group of similar users, based on the deviation assessed by the deviation assessment unit and the uncertainty assessed by the uncertainty assessment unit.

11. A physiological measurement data processing method for estimating a subjective state of mind and body of a user by using a physiological measurement data processing device including a reception unit, a deviation assessment unit, a subjective score assessment unit, an uncertainty assessment unit, and a discrepancy assessment unit, wherein
the reception unit receives, from a user terminal of the user, physiological measurement data obtained by measuring the physiological state of the user with a sensor;
the deviation assessment unit assesses a deviation of the physiological measurement data received by the reception unit with respect to past physiological measurement data of the user;
the subjective score assessment unit estimates a subjective score indicating the subjective state of mind and body of the user based on the physiological measurement data received by the reception unit;
the uncertainty assessment unit assesses an uncertainty of the subjective score estimated by the subjective score assessment unit; and
the discrepancy assessment unit assesses a discrepancy indicating a discrepancy with respect to a usual state of the user, based on the deviation assessed by the deviation assessment unit and the uncertainty assessed by the uncertainty assessment unit.

12. The physiological measurement data processing method according to claim 11, wherein the physiological measurement data processing device includes a report determination unit, and
the report determination unit performs report determination based on the discrepancy assessed by the discrepancy assessment unit.

13. The physiological measurement data processing method according to claim 12, wherein the subjective score assessment unit estimates a subjective score of the user by using a subjective score estimation model, and
the subjective score estimation model is trained by using a set of subjective score ground-truth data obtained by the user inputting the subjective state of mind and body of the user into the user terminal, and preprocessed data obtained by subjecting the physiological measurement data of the user for a period corresponding to the subjective score ground-truth data to a predetermined preprocessing.

14. The physiological measurement data processing method according to claim 13, wherein, based on the discrepancy assessed by the discrepancy assessment unit, the report determination unit determines whether or not to issue a report prompting the user to input the subjective state of mind and body of the user to the user terminal of the user.

15. The physiological measurement data processing method according to claim 13, wherein the subjective score estimation model includes a first subjective score estimation sub-model and a second subjective score estimation sub-model,
the first subjective score estimation sub-model estimates a first subjective score indicating the subjective state of mind and body of the user based on the physiological measurement data received by the reception unit,
the second subjective score estimation sub-model estimates a second subjective score indicating the subjective state of mind and body of the user based on the first subjective score, and
the subjective score assessment unit outputs the second subjective score as a subjective score to be estimated.

16. The physiological measurement data processing method according to claim 15, wherein the report determination unit determines whether or not to report the user terminal of the user of advice for improving the subjective state of mind and body, based on the discrepancy assessed by the discrepancy assessment unit.
